(19) United States
(12) Patent Application Publication
(10) Pub. No.: US 2026/0151145 A1
(43) Pub. Date: Jun. 4, 2026

(54) Navigation System Using Tracked Drill Tip for Intramedullary Nail Locking Guidance

(71) Applicant: DePuy Synthes Products, Inc., Raynham, MA (US)

(72) Inventors: Megan Greenwood, Houston, TX (US); Troy Probst, Wilmington, DE (US); Glen Pierson, Glenmoore, PA (US); Abdul Umaru, Houston, TX (US); Jorge Salazar, Houston, TX (US)

(21) Appl. No.: 19/408,501

(22) Filed: Dec. 4, 2025

Related U.S. Application Data
(60) Provisional application No. 63/727,766, filed on Dec. 4, 2024.

(51) Int. Cl. A61B 17/17; A61B 34/20; A61B 34/30; A61B 90/00

(52) U.S. Cl. CPC A61B 17/1703; A61B 17/1725; A61B 34/30; A61B 2034/2065; A61B 2034/2072; A61B 2090/365; A61B 2090/376; A61B 2090/3937; A61B 2090/3966; A61B 2090/3995

(57) ABSTRACT

A surgical system includes an instrument, tool, position markers, image sensor, and control unit. The instrument is configured to engage anatomical tissue along a trajectory that intersects an anatomical target location. The instrument's distal end engages the tissue. The tool manipulates instrument movements along the trajectory. The markers are disposed on a base surface on the tool. The image sensor is tool-mounted; defines a view field encompassing the base surface, markers, and target location; and transmits a live image stream. The control unit receives the live stream and has a processor that executes machine-readable instructions to identify visual spacing and orientation parameters of the markers in the live stream. The processor also calculates and registers in memory a reference location at the instrument's distal end in a 3D coordinate system and calculates distance offsets between the target and reference locations in the coordinate system and communicates the offsets to a user.

### CROSS REFERENCE TO RELATED APPLICATION

This application claims benefit under 35 U.S.C. § 119(e) of Provisional U.S. Patent Application No. 63/727,766, filed Dec. 4, 2024, the contents of which are hereby incorporated by reference in its entirety

### TECHNICAL FIELD

The present invention relates to surgical navigation systems that can be used in conjunction with medical devices.

### BACKGROUND

Surgical implants can include mechanisms that require external manipulation during or after implantation. For example, an implant can include anchoring elements, locking elements, position-adjusting elements, or other types of elements or features that allow the implant to operate in a manner to promote healing and/or stabilization of the anatomy of the patient. One example of such an implant includes an intramedullary (IM) nail implanted within a medullary canal of a long bone, such as a tibia, fibula, femur, and humerus, for example, to stabilize a fracture in the bone. It has been common practice to affix the IM nail with respect to the bone by placing locking members, such as screws, through pilot access holes drilled through a cortex of the bone and in alignment with locking holes, such as threaded bores, extending transversely through the IM nail. The procedure presents technical difficulties, as the locking holes in the IM nail are not generally visible to the surgeon, and are difficult to localize and to align with surgical drills and placement instruments for drilling the access holes.

### SUMMARY

According to an embodiment of the present disclosure, a surgical system includes an instrument configured to engage anatomical tissue along a trajectory that intersects a target location within an anatomical structure. A distal end of the instrument is configured to engage the anatomical tissue. The surgical system includes a tool configured to manipulate movement of the surgical instrument along the trajectory. A first array of position markers is disposed on a base surface operatively carried on the tool. An image sensor is mounted to the tool and defines a field of view that encompasses the base surface, the first array of position markers, and the target location. The image sensor is configured to transmit a live image stream for image processing. The surgical system includes a control unit configured to receive the live image stream from the image sensor. The control unit comprising a processor in communication with computer memory, wherein the processor is configured to execute machine-readable instructions stored in the computer memory to identify visual spacing and orientation parameters of the first array of position markers in the live image stream. The processor is also configured to calculate and register in the computer memory a reference location at the distal end of the surgical instrument in a three-dimensional coordinate system and to calculate distance offsets between the target location and the reference location in the three-dimensional coordinate system and communicate the distance offsets to a user.

According to another embodiment of the present disclosure, a surgical system includes a tool, first and second arrays of position markers, a camera, an X-ray images, and a control unit. The surgical instrument is configured to engage anatomical tissue along a trajectory that intersects a target location within an anatomical structure. The surgical instrument defines a distal end configured to engage the anatomical tissue. The tool is configured to manipulate movement of the surgical instrument along the trajectory. The first array of position markers is disposed on a base surface operatively carried on the tool. The second array of position markers is disposed on at least one additional base surface operatively affixed to the anatomical structure. The camera defines a first field of view and is positioned such that the first field of view encompasses the first and second arrays of position markers and the target location when the surgical instrument is coupled to the tool and oriented along the trajectory. The camera is configured to transmit a live image stream of the first field of view for image processing. The X-ray imager defines a second field of view and is position-adjustable such that the second field of view encompasses the second array of position markers. The X-ray imager is configured to transmit X-ray images for image processing. The control unit is configured to receive the live image stream and the X-ray images. The control unit has a processor configured to execute machine-readable instructions stored in computer memory to identify visual spacing and orientation parameters of the first and second arrays of position markers in the live image stream and the second array of position markers in the X-ray images. The processor is also configured to calculate and register in the computer memory a reference location at the distal end of the surgical instrument in a three-dimensional (3D) coordinate system. The processor is further configured to calculate distance offsets between the target location and the reference location in the 3D coordinate system and communicate the distance offsets to a user.

According to an additional embodiment of the present disclosure, a surgical system includes a drill bit, a surgical drill, first and second arrays of position markers, a camera, an X-ray imager, and a control unit. The drill bit has a distal end configured to engage cortical bone material of a long bone along a trajectory that intersects a target location associated with a locking hole of an intramedullary nail residing within the long bone. The surgical drill is configured to drive the drill bit along the trajectory. The first array of position markers is disposed on a first base surface operatively carried on the surgical drill. The second array of position markers is disposed on at least one additional base surface operatively affixed with respect to the long bone. The camera defines a first field of view and is positioned such that the field of view encompasses the first and second arrays of position markers and the target location when the drill bit is coupled to the surgical drill and oriented along the trajectory. The camera is configured to transmit a live image stream of the first field of view for image processing. The X-ray imager defines a second field of view and is position-adjustable such that the second field of view encompasses the second array of position markers. The X-ray imager is configured to transmit X-ray images for image processing. The control unit is configured to receive the live image stream and the X-ray images. The control unit includes a processor configured to execute machine-readable instructions stored in computer memory to identify visual spacing and orientation parameters of the first and second arrays of position markers in the live image stream and the second array of position markers in the X-ray images. The processor is also configured to calculate and register in the computer memory a reference location at the distal end of the drill bit in a three-dimensional (3D) coordinate system. The processor is further configured to calculate distance offsets between the target location and the reference location in the 3D coordinate system and communicate the distance offsets to a user.

### BRIEF DESCRIPTION OF THE DRAWINGS

The foregoing summary, as well as the following detailed description of example embodiments of the present disclosure, will be better understood when read in conjunction with the appended drawings. For the purposes of illustrating the example embodiments of the present disclosure, references to the drawings are made. It should be understood, however, that the application is not limited to the precise arrangements and instrumentalities shown. In the drawings:

FIG. 1A is a diagram view of a surgical guidance system for navigating a surgical instrument to a target location of a medical implant, according to an embodiment of the present disclosure;

FIG. 1B is a sectional end view of the medical implant with the surgical instrument positioned at a target location and oriented at a target trajectory for accessing a targeted feature of the medical implant;

FIG. 1C is a sectional end view of the medical implant, showing the surgical instrument engaged with the targeted feature of the implant;

FIG. 2A is a diagram view showing an imaging device, a surgical instrument, an instrument support member having a first array of fiducial markers, an implant, and a marker support member affixed to the implant and having a second array of fiducial markers, each of these components for use in the surgical guidance system illustrated in FIG. 1A;

FIG. 2B is a sectional end view of the surgical instrument engaged with the instrument support member, taken along section line 2B-2B in FIG. 2A;

FIG. 2C is a perspective, partial sectional view showing the second array of fiducial markers, the surgical instrument, and the implant illustrated in FIG. 2A;

FIG. 2D is a perspective view showing the marker support member having the second array of fiducial markers of FIG. 2C disposed thereon;

FIG. 2E is a diagram view showing a spatial relationship of the second array of fiducial markers and the implant illustrated in FIG. 2C;

FIG. 2F is a perspective view of an end portion of the implant that includes the target location;

FIG. 3A is a perspective view of the instrument support member of FIG. 2A having an alternative array of fiducial markers, according to another embodiment of the present disclosure;

FIG. 3B is a perspective view of a surgical instrument having an array of fiducial markers thereon, according to yet another embodiment of the present disclosure;

FIG. 4A-4G are diagram views showing steps in a method that employs features shown in FIGS. 1A-2F for calculating in real-time a position and trajectory of the surgical instrument relative to an imaging device, according to an exemplary embodiment of the present disclosure;

FIG. 5 is a front view of a split-screen visual display showing navigation graphics for guiding a surgical instrument to a target location of a medical implant, in which the split-screen includes a live image stream display having navigation graphics presented thereon for course adjustments alongside a diagram display having navigation graphics presented thereon for fine adjustments, according to an additional embodiment of the present disclosure;

FIGS. 6A-6D are front views of the diagram display of FIG. 5 showing operation of the navigation graphics;

FIG. 7 is a front view of the diagram display of FIG. 5 showing navigation graphics associated with a sequence of user movements of a tracked instrument; and

FIGS. 8A-8D are perspective views showing additional marker support members having respective arrays of fiducial markers thereon, according to additional embodiments of the present disclosure.

### DETAILED DESCRIPTION

The present disclosure can be understood more readily by reference to the following detailed description taken in connection with the accompanying figures and examples, which form a part of this disclosure. It is to be understood that this disclosure is not limited to the specific devices, methods, applications, conditions or parameters described and/or shown herein, and that the terminology used herein is for the purpose of describing particular embodiments by way of example only and is not intended to be limiting of the scope of the present disclosure. Also, as used in the specification including the appended claims, the singular forms “a,” “an,” and “the” include the plural, and reference to a particular numerical value includes at least that particular value, unless the context clearly dictates otherwise.

The term “plurality”, as used herein, means more than one. When a range of values is expressed, another embodiment includes from the one particular value and/or to the other particular value. Similarly, when values are expressed as approximations, by use of the antecedent “about,” it will be understood that the particular value forms another embodiment. All ranges are inclusive and combinable.

The terms “approximately”, “about”, and “substantially”, as used herein with respect to dimensions, angles, ratios, and other geometries, takes into account manufacturing tolerances. Further, the terms “approximately”, “about”, and “substantially” can include 10% greater than or less than the stated dimension, ratio, or angle. Further, the terms “approximately”, “about”, and “substantially” can equally apply to the specific value stated.

It should be understood that, although terms involving numerical prepositions (e.g., “first,” “second”) can be used herein to describe various features, such features should not be limited by these terms. These terms are instead used to distinguish one feature from another. For example, a first element could be termed a second element in another context, and, similarly, a second element could be termed a first element in another context, without departing from the scope of the embodiments disclosed herein.

The embodiments described herein relate to surgical guidance systems that provide navigational assistance to help a surgeon navigate a surgical instrument to a target location of an implanted medical device. Some implants, such as intramedullary (IM) nails, have target locations that are particularly challenging for a surgeon to locate during an IM nailing procedure. For example, IM nails typically have distal locking holes that are configured to receive bone anchors, such as bone screws, to affix the distal portion of the IM nail to the region of the bone in which the IM nail is implanted. However, because the distal locking holes of an IM nail reside within the medullary canal, those locking holes are obscured from the surgeon's view not only by soft tissue, but by cortical bone. Although fluoroscopic imagery can be employed to identify and target the distal locking holes of an IM nail, challenges remain, particularly because excessive fluoroscopic imagery can pose health risks to the patient. The surgical guidance systems disclosed herein are adapted to reduce the time, radiation exposure, and stress associated with locking an IM nail. The surgical guidance systems disclosed herein address these challenges by identifying the target locking hole locations, tracking the location of a surgical drill relative to the locking holes, and providing navigation graphics to help the surgeon navigate the drill tip to the location for drilling into and through the target holes. In particular, the surgical guidance systems disclosed herein are adapted to account for undesirable relative movements between the drill bit and the tracking sensors that can otherwise diminish the efficacy of a surgical navigation system. Such relative movements include instances of drill bit deflection while drilling into the cortical bone material in front of the target locking hole, and also include relative movements between the drill bit and the power tool that drives the drill bit. By accounting for such relative movements, the surgical guidance systems disclosed herein can provide enhanced navigation performance for targeting the IM nail locking holes.

It should be appreciated that, although the embodiments illustrated herein pertain to intramedullary (IM) nails, the features, principles, and concepts of the invention(s) disclosed herein can be adapted for other surgical targeting and navigation purposes, such as for bone plating procedures (e.g., targeting plate holes for bone screw insertion therein) and knee replacement surgeries, by way of non-limiting examples.

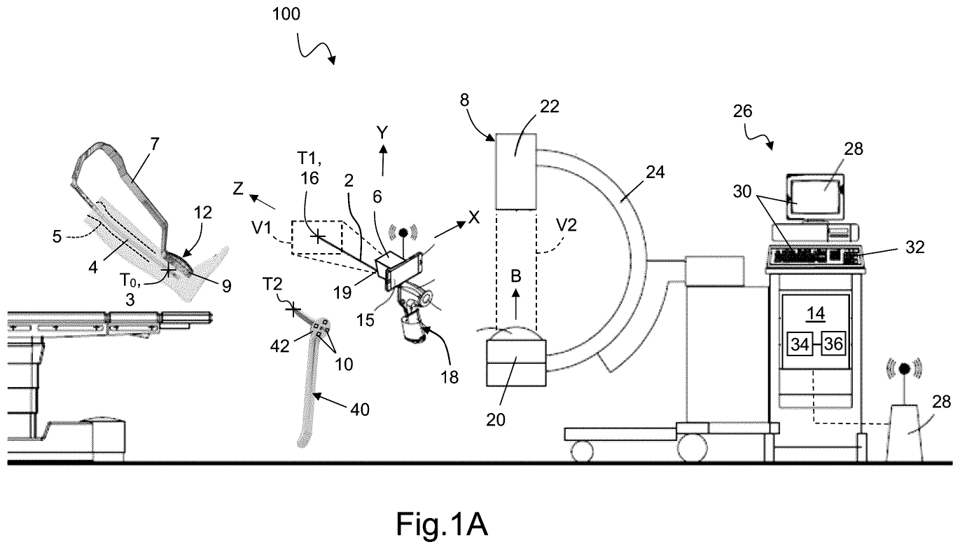

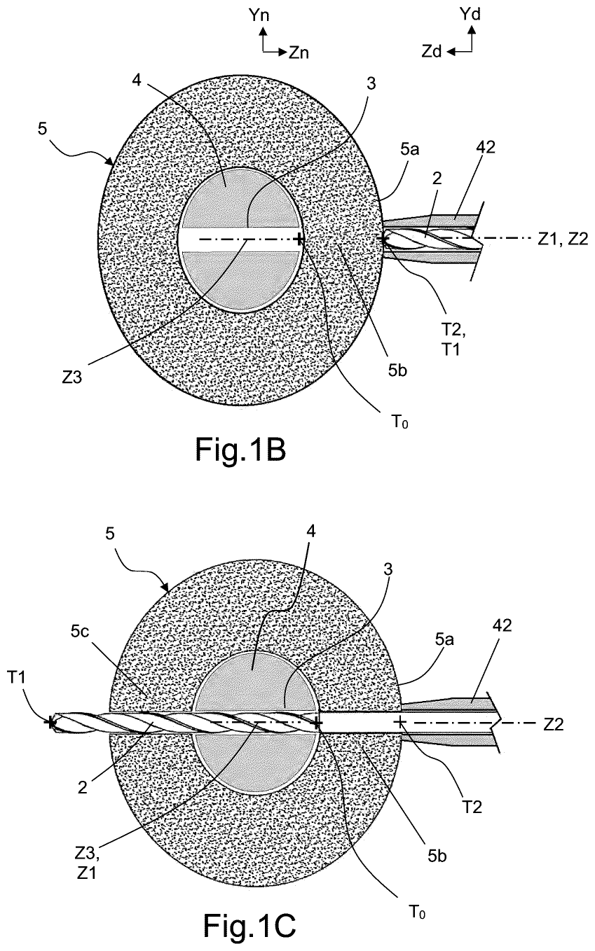

Referring now to FIG. 1A, an exemplary surgical guidance system 100 is shown for navigating a surgical instrument 2, such as a drill bit 2, to a target location T0 of a medical implant 4, such as a target locking hole 3 of an IM nail 4. In particular, the surgical guidance system 100 is configured to identify the target location T₀ within an anatomical structure 5, such as bone, and in real time track the position of the drill bit 2 relative to the target location T₀. The surgical guidance system 100 is further configured to generate and display virtual navigation graphics that indicate to the surgeon the position of the drill bit 2 relative to the target location T₀. The navigation graphics also indicate to the surgeon the desired trajectory of the drill bit 2 relative to the target location T₀. Both the target location T₀ and the trajectory are necessary to pre-drill through the locking hole 3 with the drill bit 2, as shown in FIGS. 1B-1C. In particular, the drill bit 2 is configured to drill through the bone 5 at a location and trajectory in alignment with a central hole axis Z3 of the locking hole 3. In particular, the drill bit 2 is configured to purchase against an outer surface 5a of the bone 5 at the near cortex 5b (i.e., the cortex between the locking hole 3 and the dill bit 2 along the navigated trajectory) before the drill bit 2 is driven. When confirmed that the drill bit 2 is at the foregoing position and trajectory (FIG. 1B), the drill bit 2 can be driven to drill through the near cortex 5b and advance through the locking hole 3 along the central hole axis Z3 and further into the far cortex 5c. In this manner, the drill bit 2 is configured to create pilot holes in both the near and far cortex 5b, 5c along the locking hole axis Z3, as shown in FIG. 1C. The pilot holes (particularly in the near cortex 5b) facilitate insertion of a locking screw through the near cortex 5b, through the locking hole 3, and preferably into the far cortex 5c for affixing the distal end of the IM nail 4 within the bone.

Referring again to FIG. 1A, the surgical guidance system 100 includes a first imaging device 6 for generating first image data, a second imaging device 8 for generating second image data, and a first marker array 10 locatable at a known position relative to the surgical instrument 2 and within a field of view V1 of the first imaging device 6. In this manner, the first image data includes images of the first marker array 10. The surgical guidance system 100 also includes a second marker array 12 configured to be located near the treatment site, particularly at a known position relative to the target location T₀. For example, the second marker array 12 can be disposed on a positioning member that operatively couples with the IM nail 4. In the illustrated embodiment, the second marker array 12 is disposed on a designated marker arm 9 that is attachable to a proximal handle 7 of the IM nail 4. this manner, the marker arm 9 and proximal handle 7 can substantially retain the relative position between the second marker array 12 and the IM nail 4. The second marker array 12 is configured to be within the field of view V1 of the first imaging device 6 during use. Accordingly, the first image data should include images depicting both the first and second marker arrays 10, 12.

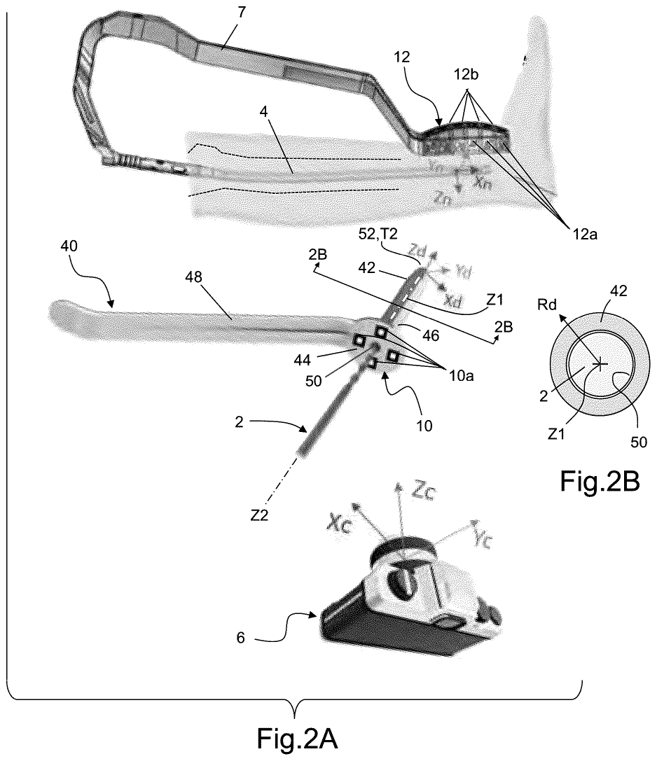

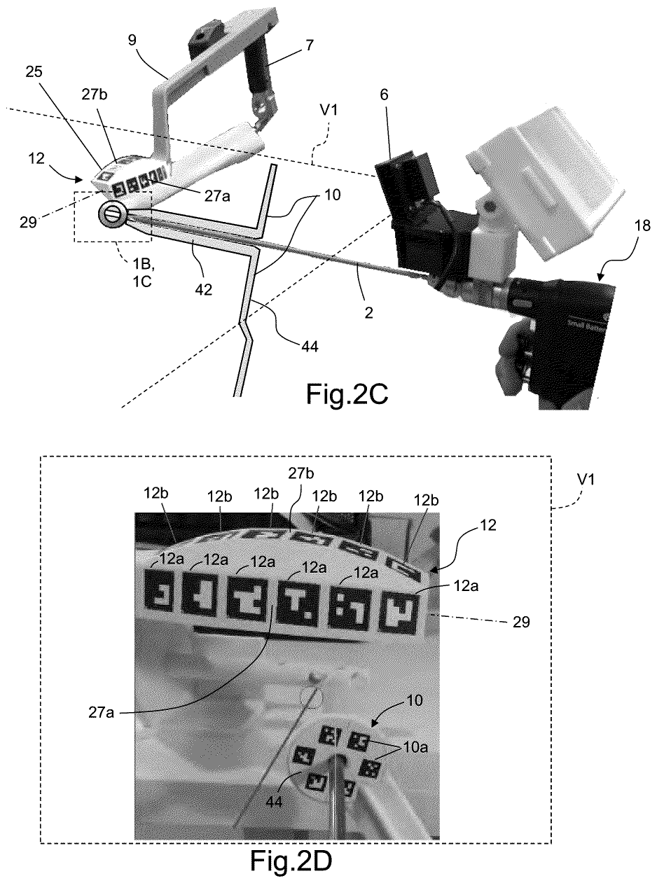

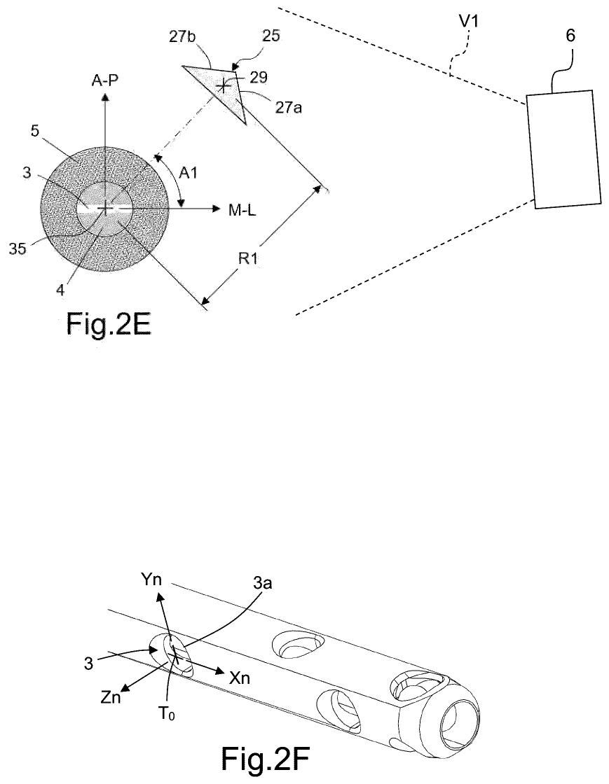

The surgical guidance system 100 includes a control unit 14 for interpreting the first and second image data and generating navigation graphics therefrom. The system 100 also includes a display device 15 for displaying the navigation graphics to a user. The first and second marker arrays 10, 12 each have one or more fiducial markers 10a, 12a,b (see FIG. 2A) that are visually distinguishable to the first imaging device 6. At least one of the first and second marker arrays 10, 12 has one or more fiducial markers that are also visually distinguishable to the second imaging device. Additionally, the one or more markers 10a, 12a,b have respective geometries that define marker keypoints or landmarks that are employed to calculate, among other things, the spatial orientation (i.e., “pose”) of the respective marker arrays 10, 12 relative to the first imaging device 6 and/or the second imaging device 8.

With continued reference to FIG. 1A, the first imaging device 6 is preferably a camera configured for generating the first image data in the form of a live image stream. The camera 6 is oriented such that the camera field of view V1 (also referred to herein as the “camera view” V1) encompasses a first reference location T1 of the drill bit 2. The first reference location T1 is preferably a distal tip 16 of the drill bit 2 spaced opposite a proximal end 19 of the drill bit 2, as shown. Accordingly, the first reference location T1 can be referred to as the “drill tip location” T1. The camera 6 defines a three-dimensional (3D) camera coordinate system Xc, Yc, Zc (see FIG. 2A), with is defined along three (3) camera coordinate axes that are perpendicular to each other and intersect each other at a camera origin location, such that the camera coordinate axes are defined by and move with the camera 6. The three (3) camera coordinate axes extend along a center beam direction Z, a lateral direction X, and a transverse direction Z, respectively, that are each perpendicular to each other by virtue of the fact that the coordinate axes are perpendicular to each other. The camera coordinate system Xc, Yc, Zc is employed as a common coordinate system for registering relevant image information in the camera view V1, including the first and second marker arrays 10, 12, the target location T0 and drill tip location T1, and additional image information described below.

The drill bit 2 can be coupled to a surgical tool 18, such as a power drill, configured to manipulate the drill bit 2 for engaging tissue at the treatment site. As shown, the camera 6 can be mounted on the surgical tool 18 and is preferably oriented so that the drill tip location T1 is substantially centered in the camera view V1, at least when the drill bit 2 is in a neutral, undeflected configuration. In embodiments where the camera 6 is mounted on the surgical tool 18, the camera 6 should preferably mounted thereon in a manner to retain its relative position with the surgical tool 18 and minimize camera 6 vibration during tool operation. Accordingly, one or more various mounting and coupling components can be employed for the camera-to-tool attachment that mitigate relative movement (e.g., vibration) between the camera 6 and the surgical tool 18 during use, such as bearings, dampeners, and the like. In other embodiments, the camera 6 can be mounted on the drill bit 2, or at least upon a bearing member, such as a journal bearing or collar, that couples to the drill bit 2 and retains its relative axial position with the drill bit 2 while maintaining a non-rotating position thereon. In such camera-on-bit embodiments, one or more structures can be employed for mitigating unwanted relative movement (e.g., vibration) between the camera 6 and the drill bit 2.

The second imaging device 8 is an X-ray imager configured for generating the second image data in the form of X-ray imagery (e.g., fluoroscopic imagery) of the treatment site. The X-ray imager 8 includes an X-ray transmitter 20 configured to transmit X-rays through patient anatomy along a beam axis B and an X-ray receiver 22 configured to receive the X-rays from the X-ray transmitter 20. Thus, X-ray imager 8 defines an X-ray field of view V2 between the X-ray transmitter 20 and receiver 22. The X-ray imager 8 includes a positioning mechanism 24, such as a robotic arm (e.g., a C-arm), configured to adjust the X-ray field of view V2 as needed to obtain X-ray images of the treatment site, particularly showing the IM nail 4 and the target location T₀ within the patient anatomy.

The surgical guidance system 100 can include an imaging station 26 that allows user-control of various components within the system 100, such as operation of the X-ray imager 8. The imaging station 26 preferably includes a station monitor 28 configured to display at least the X-ray imagery, and optionally to also display the camera live image stream. For example, the station monitor 28 can be configured to display the X-ray imagery and the camera live image stream side-by-side in a split-screen format. Additionally or alternatively, the station monitor 28 can be configured to switch between displaying the X-ray imagery and the camera live image stream, as controlled by a user. The control unit 14 can optionally be located at the imaging station 26, as shown in FIG. 1A. Alternatively, the control unit 14 can be located remote from the imaging station. For example, the control unit 14 can be mounted to the surgical tool 18, or at a location separate from both the imaging station 26 and the surgical tool 18. With continued reference to FIG. 1A, the imaging station 26 can include a user interface 30 having controls 32 for receiving user inputs for controlling one or more operations of the control unit 14. The controls 32 can include buttons, soft keys, a mouse, voice actuated controls, a touch screen, a stylus, movement of the control unit 14, visual cues (e.g., moving a hand in front of a camera), or the like. The user interface 30 can provide outputs, including visual information (e.g., via the station monitor 28), audio information (e.g., via speaker), mechanical outputs (e.g., via a vibrating mechanism), or a combination thereof. In various configurations, the user interface 30 can include the station monitor 28, a touch screen, a keyboard, a mouse, an accelerometer, a motion detector, a speaker, a microphone, a camera, a tilt sensor, or any combination thereof.

The control unit 14 includes a main processing unit or “processor” 34 in communication with computer memory 36 storing machine-readable instructions. The processor 34 is configured to receive the first image data (the camera live image stream) and the second image data (the X-ray imagery) and execute machine-readable instructions (e.g., image processing instructions) to identify the target location T₀ and drill tip location T1 and generate navigation graphics. The main processor 34, memory 36, station monitor 28, and user interface 30 are preferably in communication with each other via wired or wireless communication. It should be appreciated that any of the above components may be distributed across one or more separate devices and/or locations.

The memory 36 can store the machine-readable instructions therein that, upon execution by the main processor 34, cause the control unit 14 to perform various operations, such as the location identification, tracking, and targeting operations described herein. Depending upon the exact configuration and type of processor, the memory 34 can be volatile (such as some types of RAM), non-volatile (such as ROM, flash memory, etc.), or a combination thereof. The control unit 14 can include additional storage (e.g., removable storage and/or non-removable storage) including, but not limited to, tape, flash memory, smart cards, CD-ROM, digital versatile disks (DVD) or other optical storage, magnetic cassettes, magnetic tape, magnetic disk storage or other magnetic storage devices, universal serial bus (USB) compatible memory, or any other medium which can be used to store information and which can be accessed by the control unit 14.

The surgical guidance system 100 can include transmitter units 38 for communicating information between various components, such as between the camera 6, the X-ray imager 8, the control unit 14, the display device 15, and the imaging station 26, by way of non-limiting examples. The transmitter units 38 can be any suitable computing device configured to receive and send information, particularly images, such as the camera live image stream, the X-ray images, and the navigation graphics. Non-limiting examples of such computing devices include those found in a portable computing device, such as in a laptop, tablet, smart phone, or the like.

The second marker array 12 is preferably configured and positioned to be within the X-ray field of view V2 and also the camera view V1. Accordingly, the second marker array 12 is configured to be represented in the first image data and the second image data. The control unit 14 processes the first and second image data pertaining to the second marker array 12, in combination with the known relative position of the target location T₀ to the second marker array 12, to determine the target location T₀ and register the target location T₀ in the camera coordinate system Xc, Yc, Zc. The foregoing process for identifying and registering a target location, such as target location T₀, is more fully described in U.S. Patent Publication No. 2023/0270507 A1, published Aug. 31, 2023, and entitled “SURGICAL IMAGING AND DISPLAY SYSTEM, AND RELATED METHODS,” the entire disclosure of which is incorporated herein by this reference.

The camera 6 and the first marker array 10 are employed to locate and register the drill tip location T1 in the camera coordinate system Xc, Yc, Zc, particularly in a manner that accounts for relative movements between the drill bit 2 and the camera 6 during use, including those movements described above.

Referring now to FIGS. 1 and 2A, the first marker array 10 can be located at any of various known relative positions to the drill bit 2 that will be within the camera view V1 during use. Such positions can be spaced from or on-board the drill bit 2. For example, in the illustrated embodiment, the first marker array 10 is spaced from the drill bit 2 and located on a guide member 40, such as a drill protector, that is configured to interact with the drill bit 2. Specifically, the drill bit 2 and the drill protector 40 are configured to interact with each other such that the relative positions between the drill bit 2 and drill protector 40 (and thus also the first marker array 10 thereon) are known during such interactions. In such embodiments, the first marker array 10 is particularly located on a base surface 44 of the guide member 40 that is within the camera view V1 during interactions with the drill bit 2. In this manner, when the drill bit 2 interacts with the guide member 40 during use, the first image data will include images of the first marker array 10, which images will be registered within the camera coordinate system Xc, Yc, Zc. Moreover, when the drill bit 2 and guide member 40 interact with each other while aiming toward the target location T₀, the second marker array 12 will also be in the camera view V1.

In this example, the drill protector 40 (also referred to herein as a “drill guide” 40) includes a cannulated guide sleeve 42, a rear surface 44 at a proximal end 46 of the guide sleeve 42, and a handle member 48, which can extend outwardly from the rear surface 44 or from an intermediate portion of the guide sleeve 42. In the present embodiment, drill guide 40 can also double as a direct measuring device (DMD), such as for measuring the insertion depth of the drill bit 2 within the bone. In such embodiments, the rear surface 44 of the drill protector 40 has the first maker array 10 thereon and also doubles as a reflector plate for the DMD. Also in such embodiments, the camera 6 can also double as a visual measurement sensor that uses the reflector plate to calculate the depth measurement. As shown in FIGS. 2A-2B, the guide sleeve 42 defines a cannulation 50, that extends from the rear surface 44 to a distal end 52 of the guide sleeve 42. The distal end 52 of the guide sleeve 42 is also referred to herein as the “sleeve tip”52. The guide sleeve 42 defines a central sleeve axis Z1 extending centrally through the cannulation 50 along an axial sleeve direction Zd. In this manner, the sleeve tip 52 is spaced from the proximal sleeve end 46 along the axial sleeve direction Zd. The cannulation 50 preferably defines an inner diameter that is only marginally greater than the outer diameter of the drill bit 2, such that the portion of the drill bit 2 extending within the cannulation 50 will remain substantially straight, and thus extend substantially coaxially with the central sleeve axis Z1. In this manner, the drill guide 40 effectively retains its relative position with the portion of the drill bit 2 extending along the cannulation 50, as least with respect to a radial sleeve direction Rd orthogonal to the central sleeve axis Z1. Thus, when the drill bit 2 extends within the cannulation 50 and the drill tip T1 is aligned with the sleeve tip 52, the position of the drill tip T1 is known, both axially (i.e., along axial sleeve direction Zd) and radially (i.e., along the radial sleeve direction Rd), based on the location of the sleeve tip 52. This positional relationship between the guide sleeve 42 and the drill bit 2 allows the sleeve tip 52 to be employed as a proxy location T2 for the drill tip T1. This is beneficial for tracking the position of the drill tip T1 because the drill tip T1 is generally not visible to the camera, particularly while drilling into bone. Additionally, the foregoing positional relationship between the guide sleeve 42 and the drill bit 2 allows the central sleeve axis Z1 to be used as a proxy for a central bit axis Z2 of the drill bit 2. It should be appreciated that the proxy location T2 is preferably located on the central sleeve axis Z1 at an axial location thereof that is aligned with the distal end 52 of the guide sleeve 42 along the radial sleeve direction Rd.

Referring now to FIGS. 2C-2D, an example of first and second marker arrays 10, 12 is shown. In the present embodiment, the markers 10a of the first array 10 (which can also be referred to as “first array” markers 10a) are arranged on the base surface 44 (i.e., the rear surface 44 of the drill protector 40) in a pattern known to the control unit 14 (i.e., stored in the computer memory 36). The base surface 44 can be substantially planar, as shown, although in other embodiments the first array markers 10a can be disposed on a base surface having a three-dimensional (contoured) geometry. The first array markers 10a can be any type that are detectable by the camera 6 and capable of indicating (e.g., by virtue of their spacing and/or orientation) the position of the proxy location T2. By way of non-limiting examples, the first array markers 10 can be ArUco markers, circular dots, squares, or can have other geometries. As shown in FIGS. 2A and 2D, the first marker array 10 can be arranged in a circular pattern of three (3) or more markers 10a positioned around the rear opening of the cannulation 50, by way of one non-limiting example. By way of non-limiting examples, the first marker array 10 can include two (2), three (3), four (4), five (5), six (6), seven (7), eight (8), nine (9), or more than nine (9) markers 10a. In additional embodiments, the first marker array 10 can consist of one (1) marker 10a having sufficiently configured keypoints to allow the control unit 14 to calculate the proxy location T2 therefrom. The first array markers 10a can optionally be colored, which can make their visual detection faster and more reliable. The processor 34 is configured to interpret the camera live image stream and identify the keypoints and spacings of the first marker array 10 and use this information to calculate the proxy location T2, as described in more detail below. One added benefit to mounting the camera 6 on the tool 18 and locating the first marker array 10 on the rear surface 44 of the drill protector 40 is that the position of the first marker array 10 will exhibit less movement relative to the camera 6 during use. This allows the image processing algorithm(s) to inspect a smaller area in the camera view V1 for the first marker array 10. This can increase the first marker 10a detection speed and reduce occurrences of false detections.

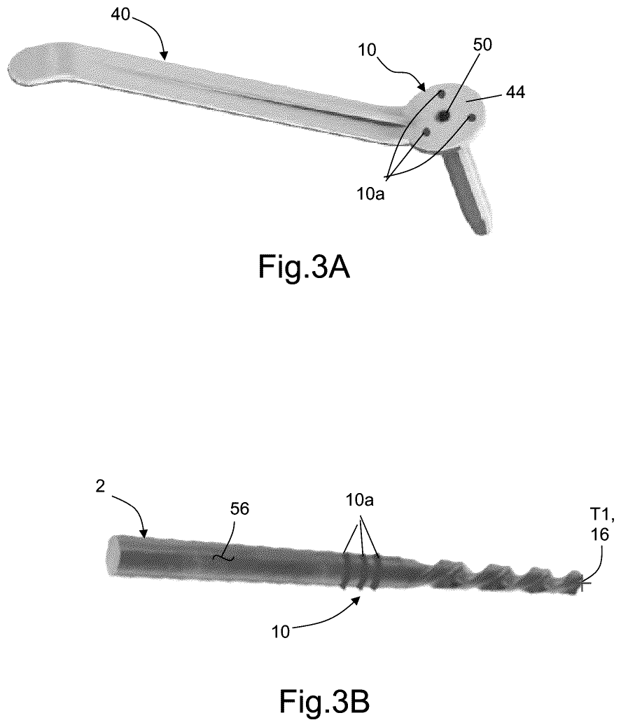

Referring now to FIG. 3A, in an additional example embodiment, the first marker array 10 can have three (3) or more circle or dot-like markers 10a on the rear surface 44 and spaced evenly around the cannulation 50. The dot-like markers 10a can be colored for faster visual detection.

Referring now to FIG. 3B, an example embodiment is shown in which the first marker array 10 is located directly on the drill bit 2. In this example, the first marker array 10 includes a plurality of annular markers 10a extending along an outer surface 54 of the drill bit 2. Because the first marker array 10 is located on the drill bit 2 in the present embodiment, the surgical guidance system 100 will use the first array markers 10a to directly track the drill tip location T1 without the need for a proxy location T2.

Referring now to FIGS. 2C-2E, the exemplary markers 12a,b of the second array 12 (which can also be referred to as “second array” markers 12a,b) are shown at a position that places them within the X-ray view V2 and also within the camera view V1 when the camera is facing the treatment site. The second array markers 12a,b are preferably ArUco markers, although other marker types can be employed, such as AprilTags developed by the APRIL Robotics Laboratory at the University of Michigan, by way of a non-limiting example. Additionally, the second array markers 12a,b are radiopaque so they can be viewed in the X-ray imagery. The marker arm 9 has a tailored geometry for placing the second array markers 12a,b close enough to the target locking hole 3 to ensure the markers 12a,b are in the camera and X-ray views V1, V2, while remaining spaced from the patient skin and out of the surgeon's way to provide an unobstructed targeting space at the treatment site. The marker arm 9 of the illustrated embodiment achieves these objectives by locating the second marker array 12 on a marker base portion 25 (also referred to herein as the “marker base” 25) that extends along a longitudinal base axis 29 that is preferably substantially parallel with the anatomical axis of the bone 5.

Additionally, the marker base 25 is configured to reside above and forward the IM nail distal locking holes 3 when the marker arm 9 is coupled to the proximal handle 7. With reference to FIG. 2E, the marker base 25 is spaced from the IM nail 4 at a radial offset distance R1 measured from the base axis 29 to a central axis 35 of the IM nail 4. The radial offset distance R1 can be in a range of about 10 mm to about 1000 mm, more preferably in a range of about 35 mm to about 100 mm, and more preferably in a range of about 45 mm to about 55 mm. Preferably, the marker base axis 29 is itself positioned at an offset angle A1 of about 45-degrees between the medial-lateral M-L and anterior-posterior A-P directions of patient anatomy during use, as shown in FIG. 2E. This places the second marker array 12 in X-ray views V2 taken at respective “perfect circle” orientations for locking holes 3 extending along the M-L direction and also for locking holes 3 extending along the A-P direction. As used herein, the term “perfect circle orientation” refers to an orientation of the IM nail 4 relative to the X-ray beam axis B at which the target hole 3 has a substantially perfectly circular shape in the X-ray imagery. It should be appreciated that, when the target hole 3 presents a perfect circle in the X-ray imagery, it indicates that the X-ray beam axis B is substantially colinear with the central hole axis Z3. Perfect circle orientation in fluoroscopy has been a common step employed in various techniques for targeting locking holes of an IM nail, including “freehand techniques,” as more fully described in U.S. Pat. No. 11,166,766, issued Nov. 9, 2021, entitled “SURGICAL INSTRUMENT MOUNTED DISPLAY SYSTEM”; U.S. Pat. No. 11,406,472, issued Aug. 9, 2022, entitled “SURGICAL INSTRUMENT MOUNTED DISPLAY SYSTEM”; and U.S. Pat. No. 11,559,359, issued Jan. 24, 2023, also entitled “SURGICAL INSTRUMENT MOUNTED DISPLAY SYSTEM,” the entire disclosure of each of which is incorporated herein by this reference. In other embodiments, the offset angle A1 can be in a range between about 0 degrees to about 90 degrees, and more particularly in a range of about 30 degrees to about 60 degrees, as measured from either the M-L direction or the A-P direction. It should be appreciated that the marker base 25 can be positioned at virtually any radial offset distance R1 and offset angle A1 that allows the second marker array 12 to be within the X-ray view V2 at both the A-P and M-L perfect circle orientations without obstructing the X-ray view V2 of the target locking hole 3. Additionally or alternatively, adaptations to the radial offset distance R1 and/or the offset angle A1 can be facilitated by the addition or reduction in the number of second array markers 12a,b on the maker base 25 and/or their orientation on the marker base 25.

In similar concept to the first array markers 10a, the second array markers 12a,b are arranged in a pattern whereby their respective orientations and keypoints (e.g., the locations of their corners) are known relative to one another. In the illustrated embodiment, the second array markers 12a,b are arranged into a first plurality of markers 12a disposed on a first base surface 27a and a second plurality of markers 12b disposed on a second base surface 27b of the marker base 25. The use of first and second distinct pluralities of markers 12a, 12b can increase the likelihood of multiple second array markers 12a,b being visible in the desired X-ray views of the target holes 3. One or both of the first and second base surfaces 27a,b can be curved, such as convex curved. Additionally, one or both of the first and second pluralities of markers 12a, 12b can be arranged in a series along the respective base surface 27a,b. As shown, the first plurality of markers 12a can be arranged in a series along the first base surface 27a, which is preferably convex curved; and the second plurality of makers 12b can be arranged in a series along the second base surface 27b, which is preferably convex curved. The first and second base surfaces 27a,b are preferably angularly offset from each other about the longitudinal base axis 29.

It should be appreciated that disposing the second array markers 12a,b on one or more curved base surfaces 27a,b provides several significant advantages for imaging purposes. One such advantage is the curved surfaces provide better detection of the marker 12a,b orientations (poses) by skewing the marker poses relative to the camera (i.e., placing the marker at a perspective view in the camera image). The inventors have observed that such skewed/perspective views of the markers 12a,b reduce image recognition difficulties typically associated with near orthogonal views of the markers. Another such advantage is that the curved surfaces provide a more robust response to lighting. For example, if the lighting causes glare on a marker 12a,b, the adjacent markers 12a,b are less likely to glare because the curved surface orients the other markers differently with respect to the light source. Yet another such advantage is that the curved surface tends to provide at least one and likely multiple of the markers 12a,b with a good pose for visibility in the camera view V1 during targeting, regardless of camera tilt or angle.

With the marker arm 9 attached to the proximal handle 7 and the second array markers 12a,b in place relative to the IM nail 4, a user can operate the C-arm 24 to take an X-ray of the IM nail 4 at a perfect circle orientation of the target hole 3. When the target hole 3 is in the perfect circle orientation and at least one (and preferably three (3) or more) of the second array markers 12a,b is visible in the X-ray view V2 and the camera view V1, the control unit 14 can use these visible second array marker(s) 12a,b to identify and register the position of the hole 3 in a common coordinate system, such as the camera coordinate system Xc, Yc, Zc. This process can be repeated for each of the target holes 3 for which navigation assistance is needed, thereby allowing their positions to be registered in the common coordinate system for targeting with the drill bit 2.

Preferably, each of the second array markers 12a,b is unique with respect to the other second array markers 12,b, and thus has a unique marker identification (ID) stored in the computer memory. When one or more of the second array markers 12a,b are visible in the X-ray image (particularly with the target hole in a perfect circle orientation), the control unit 14 will register the ID, orientation, and keypoint(s) of each visible marker(s) 12a,b relative to the target hole 3. Additionally, in the camera view V1, the control unit 14 will look for and identify any second array marker 12a,b that was visible in the X-ray view, and use that marker 12a,b (or markers 12a,b) to generate navigation graphics identifying the target location T₀. In this manner, the control unit 14 can provide the user with an accurate visualization of the target locking holes 3 of the IM nail 4 for navigated targeting with the drill tip 16.

Referring again to FIG. 2A, the first and second marker arrays 10, 12 are employed with the camera 6 to register the locations of interest, particularly the target location T₀ and the proxy location T2, into a common coordinate system, such as the camera coordinate system Xc, Yc, Zc. This allows the control unit 14 to perform spatial comparison between the target location T₀ and the proxy location T2 and to thereby generate and display navigation graphics. The first and second marker arrays 10, 12 are positioned to be within the camera view V1 so that the size and perspective of the respective marker keypoints allow the control unit 14 to calculate and track the camera orientation relative to the first and second marker arrays 10, 12 in real time. This also allows the control unit 14 to generate respective 3D coordinate systems for the target location T₀ (i.e., the locking hole 3) and the proxy location T2 (i.e., the sleeve tip 52). As shown in FIG. 2A, the proxy location T2 can be assigned a drill coordinate system Xd, Yd, Zd, with the proxy location T2 located at the origin thereof (i.e., the intersection of the coordinate axes). It should be appreciated that the axial sleeve direction Zd is the “Zd” direction in the drill coordinate system Xd, Yd, Zd. The target location T₀ can be assigned a nail coordinate system Xn, Yn, Zn, with the target location T₀ located at the origin thereof. As shown in FIG. 2F, the target location T₀ is preferably located on the central hole axis Z3 at the near opening 3a of the locking hole 3. It should be appreciated that the “Zn” direction in the nail coordinate system Xn, Yn, Zn represents the axial hole direction of the locking hole 3, that is, the direction Zn along which the central hole axis Z3 extends, as shown in FIGS. 1B-1C.

With the drill coordinate system Xd, Yd, Zd and the nail coordinate system Xn, Yn, Zn defined in the camera (common) coordinate system Xc, Yc, Zc, the spatial orientations or “poses” of the locking hole 3 (represented by the nail coordinate system Xn, Yn, Zn, with T0 as the origin thereof) and of the sleeve tip 52 (represented by the drill coordinate system Xd, Yd, Zd, with T2 at the origin thereof) relative to the camera coordinate system Xc, Yc, Zc can each be represented by a respective rotation matrix, r, and a translation matrix, t. The rotation matrix r and translation matrix for each of the locking hole 3 and the sleeve tip 52 are used to calculate a change of basis for the IM nail 4 and the guide sleeve 42 relative to the camera 6. When the poses for the camera-to-drill and for the camera-to-nail are known, changing from one coordinate system (i.e., camera coordinate system, drill coordinate system, or nail coordinate system) to another allows for obtaining consistent comparisons for x, y, and z offsets between the proxy location T2 (at the sleeve tip 52) and the target location T₀ (at the locking hole 3). Stated differently, the spatial differences between the sleeve tip 52 (and thus the drill tip 16) and the locking hole 3 can be calculated and communicated to the user for navigational purposes, both in terms of distance and trajectory. In particular, the surgical guidance system 100 is configured to communicate these spatial differences to the user via navigation graphics, as discussed in more detail below.

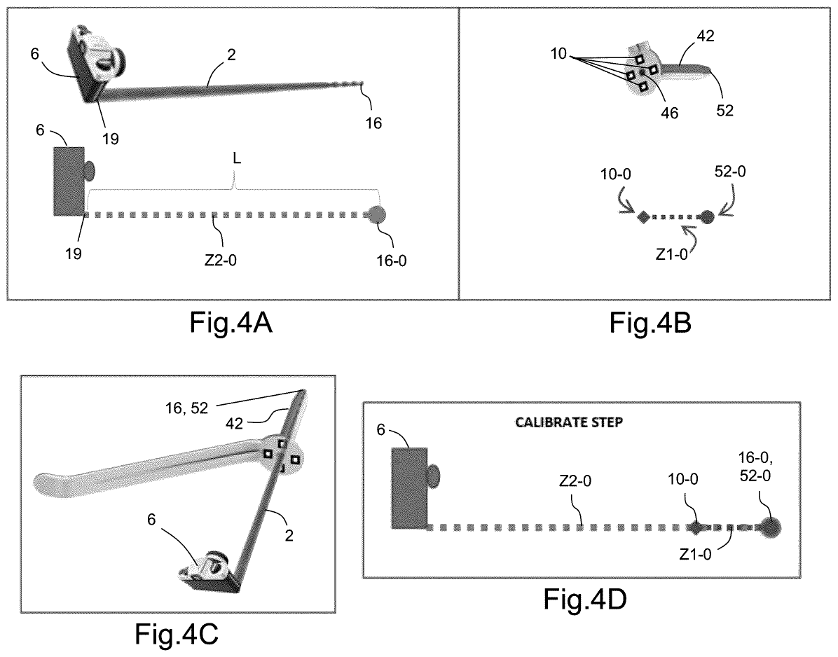

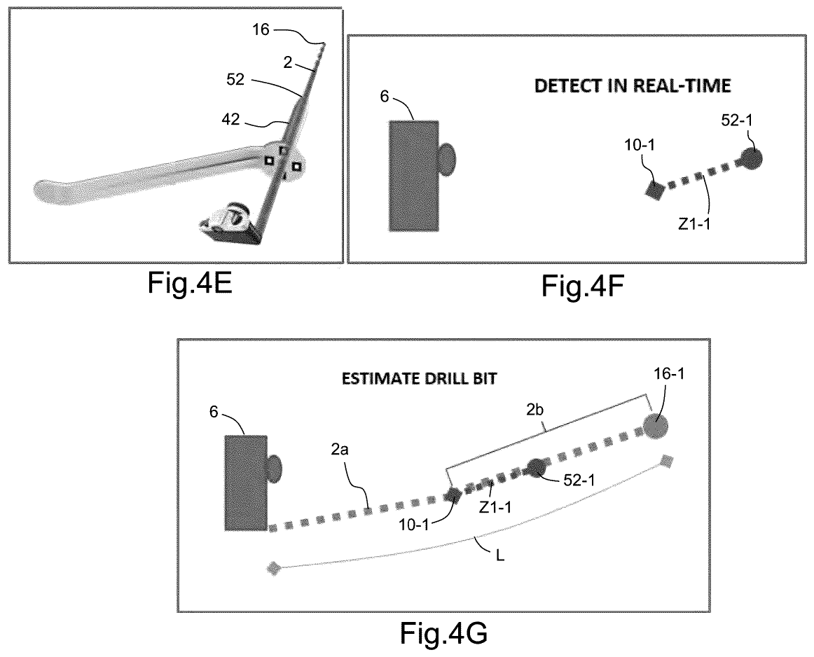

Referring now to FIGS. 4A-4G, an exemplary technique will now be described for accurately estimating the drill tip location T1 while the drill bit 2 moves distally through to the guide sleeve 42. Such movement occurs, for example, when the drill bit 2 advances through the near cortex 5b of the bone toward the target hole 3, and continues as the drill bit 2 advances through the target hole 3 and into the far cortex 5c (see FIGS. 1B-1C). The present technique, which can be referred to herein as the “sliding markers” technique, can be employed in embodiments where the camera 6 is rigidly fixed to the surgical drill 18 or axially fixed to the drill bit 2 (or at least to a non-rotating structure through which the drill bit 2 extends). Respective features and parameters that will be used for discussing in the sliding markers technique are shown in FIGS. 4A-4B.

As depicted in FIG. 4A, the camera 6 is shown attached to the drill bit 2 for illustrative purposes (although, as mentioned, above, this technique also works when the camera 6 is mounted to the surgical drill 18). The drill bit 2 is particularly shown in a neutral (undeflected) bit configuration. Also depicted in FIG. 4A is a diagram view (at bottom), showing an undeflected central bit axis Z2-0 and an undeflected length L of the drill bit 2, as measured from the proximal end 19 of the drill bit 2 to the undeflected distal tip 16-0 of the drill bit 2. It should be appreciated that, although the connection point between the drill bit 2 and the camera 6 is depicted as being coincident with the proximal end 19 of the drill bit 2, the sliding markers technique can be employed when the connection point is not coincident with the proximal end 19 of the drill bit 2 (such as when the camera 6 is mounted to the surgical drill 18). Referring now to FIG. 4B, the guide sleeve 42 is shown, with the first marker array 10 located at the proximal end 46 of the guide sleeve 42 and the sleeve tip 52 located at the distal end of the guide sleeve 42. Also depicted in FIG. 4B is a diagram view (at bottom) showing the locations of the first marker array 10-0, the predicted central sleeve axis Z1-0, and the predicted location of the sleeve tip 52-0 in a neutral sleeve position relative to the camera 6, particularly when the drill bit 2 is undeflected (i.e., straight) and the drill tip 16-0 and sleeve tip 52-0 are at the same location (i.e., when T2 serves as the proxy location for T1, as shown in FIG. 1B).

Referring now to FIGS. 4C and 4D, Step 1 of the sliding markers technique is shown. Step 1 can be referred to as the “calibration step.” At Step 1, the camera-to-marker pose is determined when the drill bit 2 is undeflected and the sleeve tip 52-0 and drill tip 16-0 are at the same location. It should also be appreciated that the connection point between the drill bit 2 and the camera 6 (or between the drill bit 2 and the surgical drill 18) can be determined during Step 1 or in a pre-calibration step before Step 1.

Step 2 is shown in FIGS. 4E and 4F. During Step 2, the drill bit 2 advances distally through the guide sleeve 42 (such as to engage bone of the near cortex 5b), and the updated camera-to-marker pose is concurrently determined in real time. The real-time updating of the camera-to-marker pose allows for the real-time calculation of both: (1) the angular change in the sleeve axis Z1 relative to the camera 6; and (2) the change in the sleeve tip location 52-1 relative to the camera 6 (e.g., the x, y, z delta in the camera coordinate system Xc, Yx, Zc).

Step 3 is shown in FIG. 4G. In Step 3, the real-time sleeve pose obtained during Step 2 is used to estimate the real-time position of the drill bit axis Z2-1. This can be performed by assuming that the drill length L remains constant even as the drill bit 2 moves or deflects. As mentioned above, the connection point between the drill bit 2 and the surgical drill 18 is known from an earlier calibration (such as during Step 1 or a pre-calibration step); additionally, the location of the first marker array 10-1 is tracked in real time via Step 2. Thus, during Step 3, a proximal segment 2a of drill bit 2 extending from connection point with the camera 6 (or with the surgical drill 18) to the location of the first marker array 10 can be modeled as a line or an arc, and the length of such line or arc can be calculated, which equates to the length of the proximal segment 2a of the drill bit 2. Subsequently, the length of a distal segment 2b of the drill bit 2 can be calculated by virtue of the fact that the total drill bit length L is known (see FIG. 4A). The distal segment 2b of the drill bit 2 can be estimated to be a straight line extending from the first marker array location 10-1 to the drill tip 16. Additionally, the central bit axis Z2 of the distal segment 2b of the drill bit 2 can be estimated to be colinear with the detected sleeve axis Z1. Using these parameters, the lengths and angular positions of the proximal and distal segments 2a,b of the drill bit 2 can be added together to calculate the position of the drill tip 16-1 relative to the camera 6 in real-time.

In an alternate calculation method for Step 3, the entire drill bit length L can be modeled as an arc that aligns with the distal and proximal ends of the sleeve (locations 10-1 and 52-1), rather than assuming the distal segment 2b of the drill bit 2 is straight. It should be appreciated that the calculations in the sliding markers technique are based on the assumptions that the connection point between the camera 6 and the drill bit 2 (or, when the camera 6 is mounted to the surgical drill 18, the camera-to-drill and bit-to-drill connection points) remain unmoved from the original calibration step.

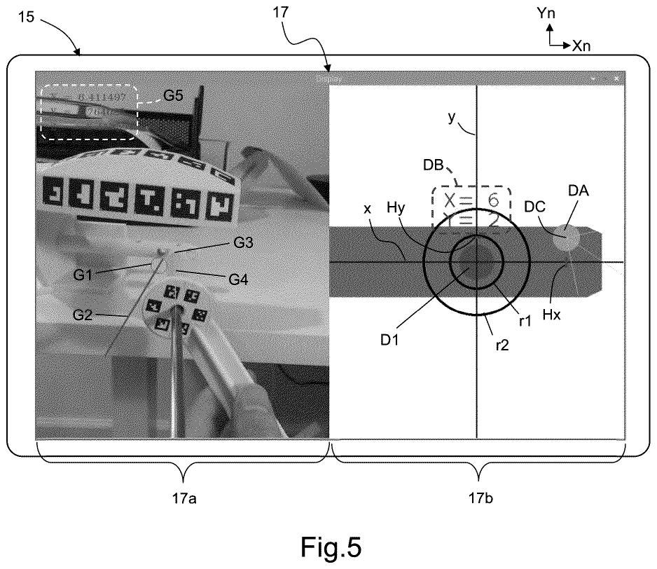

Referring now to FIGS. 5-7, examples of navigation graphics produced by the surgical guidance system 100 will now be described.

Referring now to FIG. 5, an example of a visual display 17 presented by the display device 15 (and/or by the station monitor 28) is shown. The tracking techniques described above allow the control unit 14 to generate multiple types of navigation graphics. In this example, the display device 15 is configured to present multiple types of navigation graphics in a multi-screen format, such as a split-screen format. The split-screen in the illustrated example has a first display area or screen 17a that shows the camera live image stream (or “live stream”) with virtually generated live stream navigation graphics depicted therein. Thus, the view in the live stream display 17a follows the camera view V1 and can be referred to as the “live view” 17a. The live stream can be monochrome or color. The exemplary split-screen has a second display area 17b that shows a target diagram with virtually generated diagram navigation graphics depicted therein. The second display area 17 b can be referred to as the “diagram view” 17b and is preferably fixed on the target location T₀.

The live view 17a is adapted particularly to help the user perform course positioning of the drill tip to the target location T₀. This course positioning can be performed with the drill tip 16 within an incision in the soft tissue. Additionally or alternatively, the course positioning can also aid the surgeon in selecting the location for the incision. The live navigation graphics assist the user in navigating the drill tip 16 from an ex vivo location (i.e., outside patient anatomy) to a region in close proximity to the target hole 3, such as an in vivo location, such as within an incision. For example, the live navigation graphics can include a target location graphic G1 and a target trajectory graphic G2. In the live view 17a, the target location graphic G1 can be a circular outline centered on the central hole axis Z3. As shown, the target location graphic G1 is preferably spaced from the locking hole 3 at a distance (along direction Zn) so as to be located where the central hole axis Z3 intersects the outer surface 5a of the bone 5 at the near cortex 5b, which location can be referred to as the “bone target” T_{B}. In this manner, the target location graphic G1 helps navigate the user to place the drill tip 16 at the bone target T_{B}. The target trajectory graphic G2 can be a line extending along the central hole axis Z3 in direction Zn.

The live stream navigation graphics also preferably include a tracked location graphic G3 and a tracked trajectory graphic G4. The tracked location graphic G3 is preferably superimposed at the proxy location T2 (i.e., at the sleeve tip 52). The tracked location graphic G3 can be a circle, such as a solid circle or dot. The tracked location graphic G3 preferably has a smaller diameter than the circle outline of the target location graphic G1, so that when aligned together, the tracked location dot G3 is visibly distinguishable within the target location circle G1. The tracked trajectory graphic G4 is preferably a line, such as a solid line, and can extend proximally from the tracked location dot G3 along the central sleeve axis Z1 (i.e., in direction Zd). The tracked trajectory line G4 preferably has a smaller thickness than that of the target location line G2, so that, when they extend colinearly, the tracked trajectory line G4 can be seen along and within the target trajectory line G2. Additionally, for easy visual identification for the user, the target graphics G1, G2 preferably have a different color than the tracked graphics G3, G4. For example, the target graphics G1, G2 can be red and the tracked graphics G3, G4 can be green. The live navigation graphics can also include offset readouts G5, such as in each of the such as in the X, Y, and Z directions, that indicate the current spatial offsets between the proxy location T2 and the target location T₀.

The diagram view 17b is adapted particularly to help the user make fine position adjustments for the drill bit 2. The diagram view 17b is preferably oriented in a head-on view of the target locking hole 3 using the nail coordinate system Xn, Yn, Zn. Thus, the center beam axis in the diagram view is the central nail axis Z3, i.e., along direction Zn. The diagram view 17b can also include an outline of the portion of the IM nail 4 in which target locking hole 3 resides. The diagram navigation graphics can include target graphics and tracking graphics. As with the live navigation graphics, the target graphics can have different colors than the tracking graphics. In the illustrated example, the target graphics include a main target indicator D1 or “bullseye” D1 centered at the central hole axis Z3. The bullseye D1 can be a solid circle (which can be red, for example). The target graphics can also include one or more axes, such as an x-axis and a y-axis, projected along nail directions Xn and Yn, respectively. The target graphics can further include one or more radial rings r1, r2 concentrically aligned with the bullseye D1 in target-like fashion.

The tracking graphics in the diagram view 17b preferably include a main tracking indicator DA that indicates the current position of the proxy location T2 in the Xn and Yn directions. The main tracking indicator DA can be a solid circle and can also be referred to as the “tracking bead” DA. The tracking bead DA preferably has a different color than the bullseye D1. For example, the tracking bead DA can be green while the bullseye D1 is red. Additionally, the tracking bead DA preferably has a smaller diameter than that of the bullseye D1. In this manner, when the proxy location T2 is aligned with the central hole axis Z3, the tracking bead DA will be visibly encompassed within the bullseye D1. Optionally, the tracking bead DA and the bullseye D1 can have respective sizes that are proportional to the features they visually represent (i.e, the drill bit and the locking hole, respectively). For example, if the drill bit 2 has a 4 mm diameter and the locking hole 3 has a 5 mm diameter, the tracking bead DA can have a diameter that is ⅘ that of the bullseye D1. The tracking graphics can also include hash marks Hx, Hy that track the position of the proxy location T2 along the x- and y-axes. and indicate the current position of the proxy location T2. The tracking graphics can also include offset readouts DB, such as tracked in the X and Y directions. The tracking graphics can also include a visual representation DC of the axes of the drill coordinate system (Xd, Yd, Zd). Additionally, the diagram view 17b can include an outline of the IM nail 4, which can provide the user with a sense of how the real-world movements correlate the Xn and Yn directions in the diagram view 17.

The diagram view 17b can include additional features for enhanced user experience. One such feature is involves a “snap-to-target” function, by which the tracking bead DA will automatically center in the bullseye D1 when the proxy location T2 is within an acceptable spatial tolerance of the target location T₀, such as a tolerance of +/−1 mm, by way of a non-limiting example. The inventors have observed that this snap-to-target feature helps users avoid trying to achieve perfect alignment of the drill tip 16 with the target hole 3. The snap-to-target feature can also help avoid instances where certain factors, such as the amount of image noise and/or a slow frame rate, might cause the tracking bead DA to have a “jumpy” appearance that seems to never fully land in the center of the bullseye D1. Another beneficial feature can include an auto-termination feature that “ends” the navigation phase once the drill bit is adequately centered, such as once the snap-to-target feature has occurred, unless the user specifies otherwise. This auto-terminate feature can avoid instances where the user might experience confusion while drilling into the bone because of the effect that the drilling vibrations will have on the displayed views.

The foregoing navigation graphics in the live view 17a and the diagram view 17b facilitate smooth, easy tracking and targeting for navigating the drill tip 16 to the target locking hole 3. It should be appreciated that various additional and/or alternative navigation graphics can be employed.

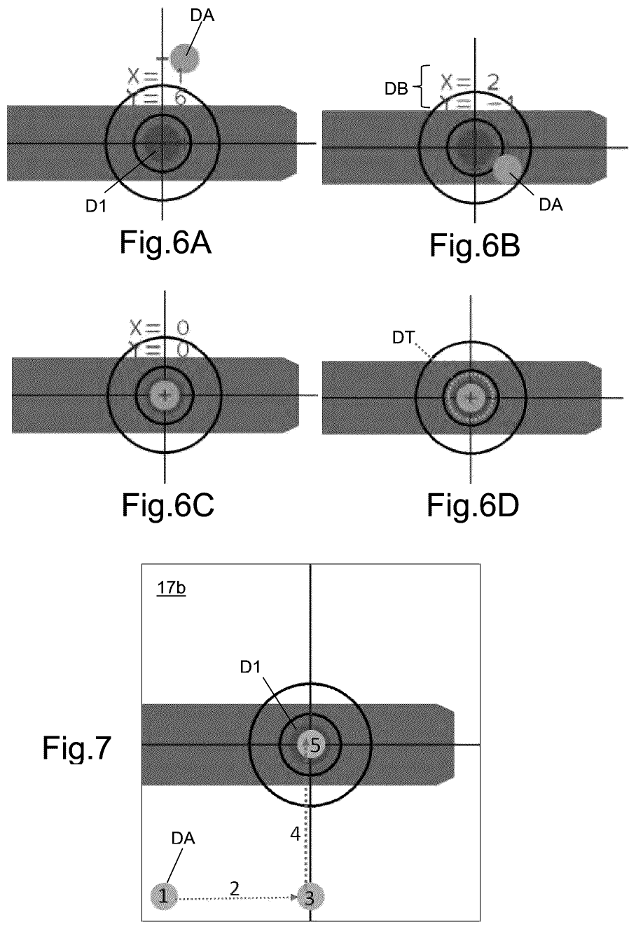

Referring now to FIGS. 6A-6D, aspects of the diagram view 17b are shown during an exemplary sequence for fine positional adjustments. In FIG. 6A, the tracking bead DA is offset from the bullseye D1 and located outside both radial rings r1, r2. In FIG. 6B, the user has brought the drill tip closer to the target hole, as demonstrated by the tracking bead DA moving to a position within the outer radial ring r2. The offset readouts DB also indicate the current offset distances in the X and Y directions as the user moves the drill tip. In FIG. 6C, the drill tip has been moved within the tolerance to activate the snap-to-target feature, which has moved the tracking bead DA within the bullseye D1. In FIG. 6D, an example tolerance threshold is indicated by a dashed circle indicator DT that is concentric with the bullseye and the tracking bead.

Referring now to FIG. 7, an exemplary workflow sequence is illustrated for using the diagram view 17b to place the drill tip (indicated by the tracking bead DA) at the target location (indicated by the bullseye D1). At stage 1, the tracking bead is positioned at a far-off distance from the bullseye, near an outer boundary of the diagram view 17b. During stage 2, the user can slide the drill tip along the outer surface of the bone in the X direction toward the y-axis until the tracking bead DA is aligned with the y-axis, which position is shown at stage 3. During stage 4, the user can slide the drill tip along the outer surface of the bone in a direction along the y-axis until the tracking bead DA is aligned with the bullseye. When the tracking bead DA is shown in the diagram view 17b to be aligned with the bullseye, the user can check that there are no significant deflections on the drill bit by moving it around and taking off any lateral loads. If the tracking bead stays centered at the bullseye during this checking process, the drill tip is adequately positions to begin drilling. If the tracking bead DA does not stay centered on the bullseye during the checking process, user can make fine adjustments to until ensured that the drill tip is adequately positioned on the target location T₀.

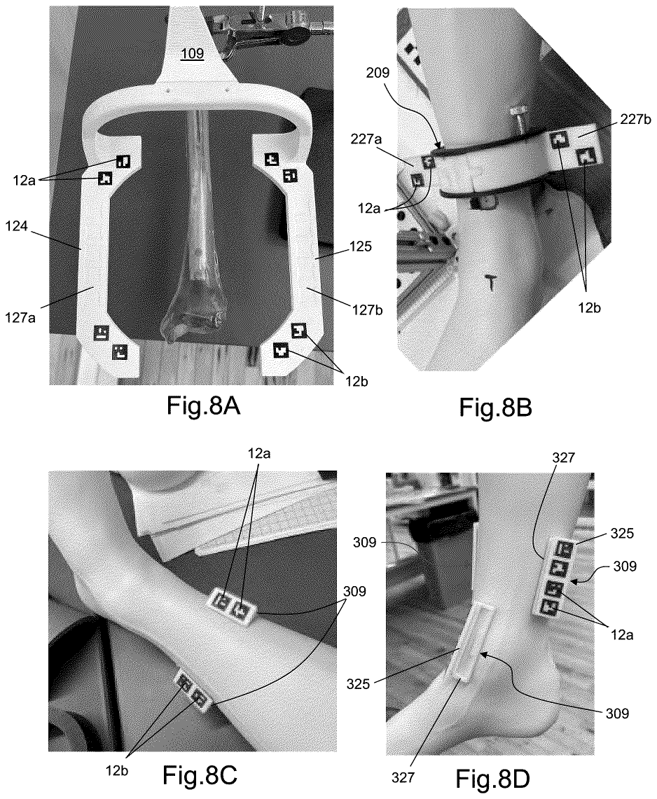

With reference to FIGS. 8A-8D, additional embodiments relating to the second marker array 12 will now be described.

As shown in FIG. 8A, the second marker array 12 can be located on a marker arm 109 having first and second base portions 124, 125 positioned on opposite sides of the bone. The base portions 124, 125 in this embodiment can have respective planar base surfaces 127a,b on which the markers 12a, 12b are disposed. In particular, a first plurality of markers 12a can be disposed on the first base surface 127a and a second plurality of markers 12b can be disposed on the second base surface 127b. The relative positions of the markers 12a, 12b can be known and recorded in the computer memory. The marker arm 109 can attach to the proximal handle 7 in simar fashion to the marker arm 9 described above.

As shown in FIG. 8B, the second marker array 12 can be located on a marker cuff 209 that can be clipped onto place around a patient limb, such as a leg into which an IM nail is implanted. The marker cuff 209 can have a pair of base surfaces 227a,b extending on opposite sides of the limb. The base surfaces 227a,b can each have one or more markers 12a,b disposed thereon at known relative positions to each other. The marker cuff 209 allow for adjustable positioning of the cuff relative to the IM nail 4. It should be appreciated that the marker cuff 209 can be made in different sizes and can have markers in-plane and/or out-of-plane with the IM nail.

As shown in FIG. 8C, the second marker array 12 can have markers 12a,b printed on an adhesive-backed substrate (a “sticker block”) 309. Such marker sticker blocks 309 allow the markers 12 to be arranged in bunches and placed directly onto the patient pre-operatively via the adhesive on the sticker block 309. The marker sticker blocks 309 allow a significant amount of flexibility in regards to final placement of the markers 12a,b.

As shown in FIG. 8D, the marker sticker blocks 309 described can include a toggle-enabled base member 325 that is coupled to a substrate member 327, such that the base member 325 can be toggled about 90-degrees relative to the substrate member 327 as desired for viewing the marker(s) in connection with M-L locking holes or A-P locking holes.

It should be appreciated that various additional marker base configurations and marker arrangements can be employed for the second array markers 12a,b.

With reference again to FIGS. 2A and 2C-2E, additional parameters for the camera 6 will now be described.

### Field of View (FOV)

The camera 6 preferably has a defined field of view (FOV) V1, which enhances the likelihood that the camera 6 captures in its field of view V1 all necessary fiducial markers 10a, 12a,b and the drill bit 2 and/or the guide sleeve 42 during navigation. It should be appreciated that the FOV requirements for the camera 6 are influenced by a relationship between (1) the position of the second marker array 12 relative to the target hole 3, and (2) the focal depth distance, which for purposes of this disclosure is defined by the distance between the drill tip 2 and the IM nail 4. In one non-limiting example embodiment, the marker base 25 places the second marker array 12 at an angular offset A1 of about 45 degrees between the M-L and A-P directions and a radial offset distance R1 of about 50 mm from the central nail axis 35. In this particular example, the camera 6 preferably has a focal depth distance in a range of about 100 mm to about 200 mm. To satisfy the foregoing objectives and parameters, the camera 6 should have a FOV of about 100 degrees and/or a field area of about 190 mm×110 mm at a drill tip-to-nail distance of 100 mm.

### Resolution and Frames Per Second (FPS)

The camera 6 has a defined image resolution that allows a user to accurately navigate the drill bit 2 to the target hole 3 without “jumpiness” or lag in the navigation graphics.

The resolution of the images provided by the camera 6 (i.e., the images in the live image stream V1) is set based on the required accuracy for the surgical navigation system 100. For this requirement, accuracy is defined as the pixel-to-pixel relationship transformed to the real-world point-to-point distance, as measured in mm. It should be appreciated that as the resolution of the image increases, more pixels will represent a specific feature (e.g., drill tip 16 or corner of fiducial marker 10 a, 12a,b). For a focal depth distance of 100 mm and a marker size of 12 mm, the navigation accuracy requirement is 0.1 mm or less (<=0.1mm) and the minimum image resolution is 720 p.

The camera resolution should also have a maximum limit to reduce the “load” on the processor 34 during image processing. Otherwise, the “load” can reduce the image processing speed, which is quantified herein using a frames per second (FPS) processing metric. It should be appreciated that the systems 100 disclosed herein has a set FPS processing speed that is dependent upon the image resolution. It is critical that the camera 6 have the capability to capture a wide range of FPS to account for the resolution dependency. By way of a non-limiting example, for a system FPS requirement of 15 FPS, the camera 6 must be able to capture at least 15 FPS (>=15 FPS). This requirement is set to avoid “jumpiness” on the navigation displays 17 during drill bit navigation. For example, the inventors have observed that when a user moves the drill with the mounted camera 6 laterally (e.g., from left to right), an FPS lower then 15 tends to result in enough jumpiness in the navigation graphics to cause the user to think that the system is behind or “lagging” because the navigation display and graphics will not synch up with their drill movements.

### Focus

The camera 6 has an “acceptable” focus over the defined focal depth distance. For this system, “acceptable” focus is utilized for the camera images to visualize all fiducial markers 10a, 12a,b and drill bit during navigation and provide the real-time spatial coordinates for navigation at the desired accuracy. It should be appreciated that the “acceptable” focus can be determined through various test models, including but not limited to USAF-1951. It should also be appreciated that the “acceptable” focus can be achieved with a fixed focus or dynamic focus (i.e., Autofocus). In an exemplary preferred embodiment, the camera 6 can have a focal depth range of 100-200 mm with fixed f-number between 5 to 6.5, a minimum relative MTF spec of approximately 0.07 cycles/pixel, is suitable for medical products, and has a “De-Bayer” image stream for viewable images.

### Size and Weight

The camera 6 should not be a visual obstruction on the surgical tool 18 or add significant weight thereto. As mentioned above, the camera 6 can be mounted on any location on the surgical tool 18 that has line of sight of the drill bit 18 and the fiducial markers 10a, 12a,b. In the embodiment shown in FIG. 2C, the camera 6 is shown mounted to the drill 18 at a fixed oblique angle relative to the drill bit axis Z2. Additionally, the camera 6 can be mounted behind a computing device 70, which can include the control unit 14. Preferably, the camera 6 is laterally centered with respect to the drill bit axis Z2 so that the camera FOV is symmetrical is symmetrical, which allows the user to change the orientation of the drill throughout the procedure while maintaining view of the markers around the patient anatomy (i.e., around the leg, such as for a tibial IM nail).

### Interface

The camera 6 is configured to provide a live image stream to a display device, such as the display device 15 and/or station monitor 28 described above. The live image stream can be either color or monochrome. The camera 6 can transfer the live image stream wirelessly or over any suitable wired connection, such as a USB connection (e.g., micro, A, C), by way of non-limiting examples. For wired connection, the connection can be an isolated cable and/or a cable built-in to the mounting structure(s).

It should be appreciated that the components, features, elements, and techniques described above can be adapted for use in other surgical indications, such as for targeting various other locations and/or features of medical implants. It should also be appreciated that, unless stated otherwise herein, the various features and aspects of the embodiments disclosed above can be adapted and incorporated into the other embodiments disclosed herein.

Various embodiments of the present disclosure can be understood in view of the following clauses:

Clause 1: a Surgical System, Comprising:
a surgical instrument configured to engage anatomical tissue along a trajectory that intersects a target location within an anatomical structure, the surgical instrument defining a distal end configured to engage the anatomical tissue;
a tool configured to manipulate movement of the surgical instrument along the trajectory;
a first array of position markers disposed on a base surface operatively carried on the tool;
an image sensor mounted to the tool, the image sensor defining a field of view that encompasses the base surface, the first array of position markers, and the target location, wherein the image sensor is configured to transmit a live image stream for image processing; and
a control unit configured to receive the live image stream from the image sensor, the control unit comprising a processor in communication with computer memory, wherein the processor is configured to execute machine-readable instructions stored in the computer memory to identify visual spacing and orientation parameters of the first array of position markers in the live image stream, the processor configured to calculate and register in the computer memory a reference location at the distal end of the surgical instrument in a three-dimensional coordinate system, the processor further configured to calculate distance offsets between the target location and the reference location in the three-dimensional coordinate system and communicate the distance offsets to a user.

Clause 2: The surgical system of clause 1, further comprising a display device, wherein the processor is configured to generate and display navigation graphics indicating the distance offsets on the display device.

Clause 3: The surgical system of clause 2, wherein the display device is mounted to the tool.

Clause 4: The surgical system of clause 2, wherein the display device is remote from the tool.

Clause 5: The surgical system of clause 2, wherein the navigation graphics are superimposed on a display of the live image stream on the display device.

Clause 6: The surgical system of clause 1, comprising a second array of position markers disposed on at least one additional base surface operatively affixed to the anatomical structure, wherein the second array of position markers is substantially encompassed within the field of view when the surgical instrument is oriented along the trajectory.

Clause 7: The surgical system of clause 6, further comprising an X-ray imager having a robotic positioning arm, wherein the second array of position markers are radiopaque for visual depiction in X-ray images produced by the X-ray imager.

Clause 8: The surgical system of clause 7, wherein the second array of position markers are visually depictable in the X-ray images and in the live image stream, wherein the processor is configured to execute the machine-readable instructions to identify visual spacing and orientation parameters of the second array of position markers in the X-ray images and in the live image stream and calculate and register in the computer memory target coordinates in the three-dimensional coordinate system, wherein the processor uses the target coordinates to calculate the distance offsets.

Clause 9: The surgical system of clause 8, wherein the second array of position markers comprise a plurality of ArUco markers.

Clause 10: The surgical system of clause 8, wherein the at least one additional base surface is convex curved, such that each of the position markers in the second array of position markers are non-coplanar with each other.

Clause 11: The surgical system of clause 8, wherein the target location is defined by a medical implant residing within the anatomical structure, and the at least one additional base surface is supported by a support member coupled to the implant.

Clause 12: The surgical system of clause 11, wherein the medical implant is an intramedullary nail, the anatomical structure is a long bone, and the target location is associated with a target locking hole extending transversely through the intramedullary nail.

Clause 13: The surgical system of clause 12, wherein the support member extends from a handle member that is coupled to an end of the intramedullary nail.

Clause 14: The surgical system of clause 13, wherein the target locking hole extends transversely through the intramedullary nail along one of a medial-lateral direction or an anterior-posterior direction of patient anatomy, and the second array of position markers is affixed at an orientation having an angular offset in a range of about 30 mm to about 60 mm from the medial-lateral direction and the anterior-posterior direction.

Clause 15: The surgical system of clause 14, wherein the angular offset is about 45 degrees.

Clause 16: The surgical system of clause 14, wherein the second array of position markers is affixed at a radial distance in a range of about 35 mm to about 100 mm from a central axis of the intermedullary nail.

Clause 17: The surgical system of clause 16, wherein the radial distance is in a range of about 45 mm to about 55 degrees.

Clause 18: The surgical system of clause 1, wherein the surgical instrument is a drill bit elongate along a central drill axis, and the tool is a powered drill.

Clause 19: The surgical system of clause 18, further comprising a guide sleeve defining the base surface and a cannulation, the cannulation extending along a central guide axis, wherein the drill bit is configured to extend through the cannulation, such that the cannulation retains a portion of the drill bit in a substantial coaxial relationship with the cannulation.

Clause 20: The surgical system of clause 19, wherein the reference location is correlated with a proxy location, the proxy location is located on the central guide axis at an axial position aligned with a distal end of the guide sleeve along a radial direction that intersects the central guide axis and is perpendicular to the central guide axis, and the processor is configured to calculate the distance offsets in real time between the target location and the reference location based at least in part upon changes in a spatial orientation of the first array of position markers relative to the image sensor in the three-dimensional coordinate system.

Clause 21: The surgical system of clause 19, wherein the first array of position markers are ArUco markers.

Clause 22: The surgical system of clause 19, wherein the first array of position markers are circular dots.

Clause 23: The surgical system of clause 19, wherein the position markers of the first array of position markers are spaced along the base surface at angular intervals about the guide axis.

Clause 24: The surgical system of clause 19, wherein the first array of position markers are annular markers extending along an outer surface of the drill bit.

Clause 25: The surgical system of clause 1, wherein the image sensor is a camera, the field of view of the camera is a defined field of view, and the camera has a focal depth distance in a range of about 100 mm to about 200 mm.

Clause 26: The surgical system of clause 25, wherein the camera has a minimum image resolution of at least about 720 p.

Clause 27: The surgical system of clause 25, wherein the camera is configured to capture at least fifteen frames per second (FPS).

Clause 28: A surgical system, comprising:
a surgical instrument configured to engage anatomical tissue along a trajectory that intersects a target location within an anatomical structure, the surgical instrument defining a distal end configured to engage the anatomical tissue;
a tool configured to manipulate movement of the surgical instrument along the trajectory;
a first array of position markers disposed on a base surface operatively carried on the tool;
a second array of position markers disposed on at least one additional base surface operatively affixed to the anatomical structure;
a camera defining a first field of view, wherein the camera is positioned such that the first field of view encompasses the first and second arrays of position markers and the target location when the surgical instrument is coupled to the tool and oriented along the trajectory, wherein the camera is configured to transmit a live image stream of the first field of view for image processing;
an X-ray imager defining a second field of view, wherein the X-ray imager is position-adjustable such that the second field of view encompasses the second array of position markers, and the X-ray imager is configured to transmit X-ray images for image processing; and
a control unit configured to receive the live image stream and the X-ray images, the control unit comprising a processor configured to execute machine-readable instructions stored in computer memory to identify visual spacing and orientation parameters of the first and second arrays of position markers in the live image stream and the second array of position markers in the X-ray images, the processor configured to calculate and register in the computer memory a reference location at the distal end of the surgical instrument in a three-dimensional (3D) coordinate system, the processor further configured to calculate distance offsets between the target location and the reference location in the three-dimensional coordinate system and communicate the distance offsets to a user.

Clause 29: The surgical system of clause 28, further comprising a display device, wherein the processor is configured to generate and display navigation graphics indicating the distance offsets on the display device.

Clause 30: The surgical system of clause 29, wherein the display device is configured to display at least one of:
the live image stream, wherein a first subset of the navigation graphics is superimposed on the live image stream; and
a two-dimensional (2D) diagram that encompasses the target location, the 2D diagram view is taken along a view plane that is orthogonal to a view axis that is coaxial with the trajectory, wherein a second subset of the navigation graphics is superimposed on the diagram view.

Clause 31: The surgical system of clause 30, wherein the first subset of navigation graphics includes:
a first graphic (G1) that is centered on the target location in the live image stream; and
a second graphic (G2) that is colinear with a target trajectory line for the surgical instrument in the live image stream, wherein the target trajectory line intersects the target location.

Clause 32: The surgical system of clause 31, wherein the first subset of navigation graphics includes:
a third graphic (G3) that is centered on a tracked position of the reference location in the live image stream; and
a fourth graphic (G4) that is colinear with a tracked trajectory line of the surgical instrument in the live image stream.

Clause 33: The surgical system of clause 30, wherein the second subset of navigation graphics includes:
a target indicator (D1) that is centered on the target location in the 2D diagram view; and
a tracking indicator (DA) that is centered on a tracked position of the reference location in the 2D diagram view is colinear with a target trajectory line for the surgical instrument in the live image stream, wherein the target trajectory line intersects the target location.

Clause 34: The surgical system of clause 33, wherein the target indicator includes a central dot and one or more concentric rings that are spaced radially outward from the central dot.

Clause 35: The surgical system of clause 33, wherein the second subset of navigation graphics includes a distance tolerance threshold indicator (DT), and the distance tolerance threshold indicator (DT) is a circle having a radius equivalent to a pre-determined distance tolerance in a radial direction perpendicular to the view axis.

Clause 36: The surgical system of clause 35, wherein the second subset of navigation graphics includes a snap-to-target feature, such that the tracking indicator (DA) snaps centrally to the target indicator (D1) when the tracked position of the reference location is within the pre-determined distance tolerance.

Clause 37: The surgical system of clause 33, wherein the second subset of navigation graphics includes distance offset readouts (DB) in respective first and second coordinate axes that are perpendicular to each other and are both parallel and coextensive in the view plane, wherein the distance offset readouts both have a zero-value at an intersection of the first and coordinate second axes.

Clause 38: The surgical system of clause 37, wherein second subset of navigation graphics includes axis lines (x, y) indicating the first and second coordinate axes.

Clause 39: The surgical system of clause 38, wherein the second subset of navigation graphics include first and second hash marks (Hx, Hy) that track the axial position of the reference location along the first and coordinate second axes, respectively.

Clause 40: The surgical system of clause 30, wherein the display device is configured to display the live image screen and the diagram view concurrently in a split-screen format.

Clause 41: The surgical system of clause 30, wherein the display device is configured to allow viewer selection between:
displaying the live image stream in full-screen format;
displaying the 2D diagram view in full-screen format; and
displaying the live image screen and the 2D diagram view concurrently in a split-screen format.

Clause 42: A surgical system, comprising:
a drill bit having a distal end configured to engage cortical bone material of a long bone along a trajectory that intersects a target location associated with a locking hole of an intramedullary nail residing within the long bone;
a surgical drill configured to drive the drill bit along the trajectory;
a first array of position markers disposed on a first base surface operatively carried on the surgical drill;
a second array of position markers disposed on at least one additional base surface operatively affixed with respect to the long bone;
a camera defining a first field of view, wherein the camera is positioned such that the field of view encompasses the first and second arrays of position markers and the target location when the drill bit is coupled to the surgical drill and oriented along the trajectory, wherein the camera is configured to transmit a live image stream of the first field of view for image processing;
an X-ray imager defining a second field of view, wherein the X-ray imager is position-adjustable such that the second field of view encompasses the second array of position markers, and the X-ray imager is configured to transmit X-ray images for image processing; and
a control unit configured to receive the live image stream and the X-ray images, the control unit comprising a processor configured to execute machine-readable instructions stored in computer memory to identify visual spacing and orientation parameters of the first and second arrays of position markers in the live image stream and the second array of position markers in the X-ray images, the processor configured to calculate and register in the computer memory a reference location at the distal end of the drill bit in a three-dimensional (3D) coordinate system, the processor further configured to calculate distance offsets between the target location and the reference location in the three-dimensional coordinate system and communicate the distance offsets to a user.

Clause 43: The surgical system of clause 42, further comprising a guide sleeve defining the first base surface and a cannulation, the cannulation extending along a central guide axis, wherein the drill bit is configured to extend through the cannulation, such that the cannulation retains a portion of the drill bit in a substantial coaxial relationship with the cannulation.

Clause 44: The surgical system of clause 43, wherein the position markers of the first array of position markers are spaced along the first base surface at angular intervals about the guide axis.

Clause 45: The surgical system of clause 43, wherein the reference location is correlated with a proxy location, the proxy location is located on the central guide axis at an axial position aligned with a distal end of the guide sleeve along a radial direction that intersects the central guide axis and is perpendicular to the central guide axis, and the processor is configured to calculate the distance offsets in real time between the target location and the reference location based at least in part upon changes in a spatial orientation of the first array of position markers relative to the image sensor in the three-dimensional coordinate system.

Clause 46: The surgical system of clause 42, further comprising a handle member that is coupled to an end of the intramedullary nail, wherein the at least one additional base surface is supported by a support member that extends from the handle member.

Clause 47: The surgical system of clause 46, wherein the support member comprises a first marker arm and a second marker arm that are spaced from each other in one of a medial-lateral direction or an anterior-posterior direction of patient anatomy, such that the first and second marker arms extend alongside and are spaced from opposite sides of the intramedullary nail, the at least one additional base surface comprises a second base surface defined on the first marker arm and a third base surface defined on the second marker arm, wherein one or more markers of the second array of position markers are disposed on the second base surface, and one or more additional markers of the second array of position markers are disposed on the third base surface.

Clause 48: The surgical system of clause 42, wherein the at least one additional base surface comprises a second base surface and a third base surface both defined on a cuff member, the cuff member is configured to attached around a patient limb that contains the long bone, wherein the first and second base surfaces are positioned opposite each other so as to be located on opposite sides of the patient limb, wherein one or more markers of the second array of position markers are disposed on the second base surface, and one or more additional markers of the second array of position markers are disposed on the third base surface.

Clause 49: The surgical system of clause 42, wherein the position markers of the second array of position markers are printed on one or more adhesive-backed substrates that are disposed on the at least one additional base surface, and the at least one additional base surface is defined by at least one base member that is locatable directly onto patient anatomy that contains the long bone.

Clause 50: The surgical system of clause 49, wherein the at least one base member is coupled to at least one respective substrate member, and the at least one base member is configured to toggle about 90 degrees with respect to the substrate member.

While example embodiments of devices for executing the disclosed techniques are described herein, the underlying concepts can be applied to any control unit, computing device, processor, or system capable of communicating and presenting information as described herein. The various techniques described herein can be implemented in connection with hardware or software or, where appropriate, with a combination of both. Thus, the methods and apparatuses described herein can be implemented, or certain aspects or portions thereof, can take the form of program code (i.e., instructions) embodied in tangible non-transitory storage media, such as floppy diskettes, CD-ROMs, hard drives, or any other machine-readable storage medium (computer-readable storage medium), wherein, when the program code is loaded into and executed by a machine, such as a computer, the machine becomes an apparatus for performing the techniques described herein. In the case of program code execution on programmable computers, the computing device will generally include a processor, a storage medium readable by the processor (including volatile and non-volatile memory and/or storage elements), at least one input device, and at least one output device, for instance a display. The display can be configured to display visual information. For instance, the displayed visual information can include fluoroscopic data such as X-ray images, fluoroscopic images, orientation screens, or computer-generated visual representations.

The program(s) can be implemented in assembly or machine language, if desired. The language can be a compiled or interpreted language, and combined with hardware implementations.

The techniques described herein also can be practiced via communications embodied in the form of program code that is transmitted over some transmission medium, such as over electrical wiring or cabling, through fiber optics, or via any other form of transmission. When implemented on a general-purpose processor, the program code combines with the processor to provide a unique apparatus that operates to invoke the functionality described herein. Additionally, any storage techniques used in connection with the techniques described herein can invariably be a combination of hardware and software.

While the techniques described herein can be implemented and have been described in connection with the various embodiments of the various figures, it is to be understood that other similar embodiments can be used or modifications and additions can be made to the described embodiments without deviating therefrom. For example, it should be appreciated that any steps disclosed above can be performed in the order set forth above, or in any other order as desired. Further, one skilled in the art will recognize that the techniques described in the present application may apply to any communication environment, whether wired or wireless, and may be applied to any number of such devices connected via a communications network and interacting across the network. Therefore, the techniques described herein should not be limited to any single embodiment, but rather should be construed in breadth and scope in accordance with the appended claims.

What is claimed:

1. A surgical system, comprising:
a surgical instrument configured to engage anatomical tissue along a trajectory that intersects a target location within an anatomical structure, the surgical instrument defining a distal end configured to engage the anatomical tissue;
a tool configured to manipulate movement of the surgical instrument along the trajectory;
a first array of position markers disposed on a base surface operatively carried on the tool;
an image sensor mounted to the tool, the image sensor defining a field of view that encompasses the base surface, the first array of position markers, and the target location, wherein the image sensor is configured to transmit a live image stream for image processing; and
a control unit configured to receive the live image stream from the image sensor, the control unit comprising a processor in communication with computer memory, wherein the processor is configured to execute machine-readable instructions stored in the computer memory to identify visual spacing and orientation parameters of the first array of position markers in the live image stream, the processor configured to calculate and register in the computer memory a reference location at the distal end of the surgical instrument in a three-dimensional coordinate system, the processor further configured to calculate distance offsets between the target location and the reference location in the three-dimensional coordinate system and communicate the distance offsets to a user.

2. The surgical system of claim 1, further comprising a display device, wherein the processor is configured to generate and display navigation graphics indicating the distance offsets on the display device.

3. The surgical system of claim 2, wherein the navigation graphics are superimposed on a display of the live image stream on the display device.

4. The surgical system of claim 1, comprising a second array of position markers disposed on at least one additional base surface operatively affixed to the anatomical structure, wherein the second array of position markers is substantially encompassed within the field of view when the surgical instrument is oriented along the trajectory.

5. The surgical system of claim 4, further comprising an X-ray imager having a robotic positioning arm, wherein the second array of position markers are radiopaque for visual depiction in X-ray images produced by the X-ray imager.

6. The surgical system of claim 5, wherein the second array of position markers are visually depictable in the X-ray images and in the live image stream, wherein the processor is configured to execute the machine-readable instructions to identify visual spacing and orientation parameters of the second array of position markers in the X-ray images and in the live image stream and calculate and register in the computer memory target coordinates in the three-dimensional coordinate system, wherein the processor uses the target coordinates to calculate the distance offsets.

7. The surgical system of claim 6, wherein the second array of position markers comprise a plurality of ArUco markers.

8. The surgical system of claim 6, wherein the at least one additional base surface is convex curved, such that each of the position markers in the second array of position markers are non-coplanar with each other.

9. The surgical system of claim 6, wherein the target location is defined by a medical implant residing within the anatomical structure, and the at least one additional base surface is supported by a support member coupled to the implant.

10. The surgical system of claim 9, wherein the medical implant is an intramedullary nail, the anatomical structure is a long bone, and the target location is associated with a target locking hole extending transversely through the intramedullary nail.

11. The surgical system of claim 10, wherein the support member extends from a handle member that is coupled to an end of the intramedullary nail.

12. The surgical system of claim 11, wherein the target locking hole extends transversely through the intramedullary nail along one of a medial-lateral direction or an anterior-posterior direction of patient anatomy, and the second array of position markers is affixed at an orientation having an angular offset in a range of about 30 mm to about 60 mm from the medial-lateral direction and the anterior-posterior direction.

13. The surgical system of claim 12, wherein the angular offset is about 45 degrees.

14. A surgical system, comprising:
a surgical instrument configured to engage anatomical tissue along a trajectory that intersects a target location within an anatomical structure, the surgical instrument defining a distal end configured to engage the anatomical tissue;
a tool configured to manipulate movement of the surgical instrument along the trajectory;
a first array of position markers disposed on a base surface operatively carried on the tool;
a second array of position markers disposed on at least one additional base surface operatively affixed to the anatomical structure;
a camera defining a first field of view, wherein the camera is positioned such that the first field of view encompasses the first and second arrays of position markers and the target location when the surgical instrument is coupled to the tool and oriented along the trajectory, wherein the camera is configured to transmit a live image stream of the first field of view for image processing;
an X-ray imager defining a second field of view, wherein the X-ray imager is position-adjustable such that the second field of view encompasses the second array of position markers, and the X-ray imager is configured to transmit X-ray images for image processing; and
a control unit configured to receive the live image stream and the X-ray images, the control unit comprising a processor configured to execute machine-readable instructions stored in computer memory to identify visual spacing and orientation parameters of the first and second arrays of position markers in the live image stream and the second array of position markers in the X-ray images, the processor configured to calculate and register in the computer memory a reference location at the distal end of the surgical instrument in a three-dimensional (3D) coordinate system, the processor further configured to calculate distance offsets between the target location and the reference location in the three-dimensional coordinate system and communicate the distance offsets to a user.

15. The surgical system of claim 14, further comprising a display device, wherein the processor is configured to generate and display navigation graphics indicating the distance offsets on the display device.

16. The surgical system of claim 15, wherein the display device is configured to display at least one of:
the live image stream, wherein a first subset of the navigation graphics is superimposed on the live image stream; and
a two-dimensional (2D) diagram that encompasses the target location, the 2D diagram view is taken along a view plane that is orthogonal to a view axis that is coaxial with the trajectory, wherein a second subset of the navigation graphics is superimposed on the diagram view.

17. A surgical system, comprising:
a drill bit having a distal end configured to engage cortical bone material of a long bone along a trajectory that intersects a target location associated with a locking hole of an intramedullary nail residing within the long bone;
a surgical drill configured to drive the drill bit along the trajectory;
a first array of position markers disposed on a first base surface operatively carried on the surgical drill;
a second array of position markers disposed on at least one additional base surface operatively affixed with respect to the long bone,
a camera defining a first field of view, wherein the camera is positioned such that the field of view encompasses the first and second arrays of position markers and the target location when the drill bit is coupled to the surgical drill and oriented along the trajectory, wherein the camera is configured to transmit a live image stream of the first field of view for image processing; and
an X-ray imager defining a second field of view, wherein the X-ray imager is position-adjustable such that the second field of view encompasses the second array of position markers, and the X-ray imager is configured to transmit X-ray images for image processing;
a control unit configured to receive the live image stream and the X-ray images, the control unit comprising a processor configured to execute machine-readable instructions stored in computer memory to identify visual spacing and orientation parameters of the first and second arrays of position markers in the live image stream and the second array of position markers in the X-ray images, the processor configured to calculate and register in the computer memory a reference location at the distal end of the drill bit in a three-dimensional (3D) coordinate system, the processor further configured to calculate distance offsets between the target location and the reference location in the three-dimensional coordinate system and communicate the distance offsets to a user.

18. The surgical system of claim 17, further comprising a guide sleeve defining the first base surface and a cannulation, the cannulation extending along a central guide axis, wherein the drill bit is configured to extend through the cannulation, such that the cannulation retains a portion of the drill bit in a substantial coaxial relationship with the cannulation.

19. The surgical system of claim 18, wherein the position markers of the first array of position markers are spaced along the first base surface at angular intervals about the guide axis.

20. The surgical system of claim 18, wherein the reference location is correlated with a proxy location, the proxy location is located on the central guide axis at an axial position aligned with a distal end of the guide sleeve along a radial direction that intersects the central guide axis and is perpendicular to the central guide axis, and the processor is configured to calculate the distance offsets in real time between the target location and the reference location based at least in part upon changes in a spatial orientation of the first array of position markers relative to the image sensor in the three-dimensional coordinate system.
